(19) United States
(12) Patent Application Publication
(10) Pub. No.: US 2019/0046075 A1
(43) Pub. Date: Feb. 14, 2019

(54) VOLUMETRIC CAPNOGRAPHY

(71) Applicant: ORIDION MEDICAL 1987 LTD., JERUSALEM (IL)

(72) Inventor: Boaz Shay Shalev, Jerusalem District (IL)

(21) Appl. No.: 16/056,839

(22) Filed: Aug. 7, 2018

Related U.S. Application Data
(60) Provisional application No. 62/545,357, filed on Aug. 14, 2017.

(51) Int. Cl. A61B 5/083; A61B 5/097; A61M 16/06

(52) U.S. Cl. CPC A61B 5/0836; A61B 5/097; A61B 5/082; A61B 5/0833; A61M 16/06

(57) ABSTRACT

A capnographic system, the system comprising one or more processors configured to: (i) receive an initial capnographic measurement from a breath monitoring device, at least when the breath monitoring device is attached to a patient, (ii) receive a primary value of at least one attribute, other than the initial capnographic measurement, characteristic of the patient, (iii) assign, from a database containing at least one set of secondary values corresponding to primary values of the at least one attribute, a secondary value corresponding to the primary value of the at least one attribute, and (iv) calculating, based on a combination of the initial capnographic measurement and the secondary value, a refined capnographic measurement.

### TECHNOLOGICAL FIELD

The present disclosure relates generally to capnography, and more particularly, to systems and methods for verifying capnographic measurements.

### BACKGROUND

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Capnographic monitoring systems usually include a mask or a sensor attached to a patient and configured for measuring the level of carbon dioxide exhaled by the patient, and a system for receiving, displaying, and analyzing the measurements in order to deduce or identify different medical conditions of the patient.

Some capnographic monitoring systems are configured to issue an alert when the measured level of carbon dioxide exceeds a predetermined threshold or when the breathing pattern detected by the system displays abnormalities or is different than the expected breathing pattern.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In accordance with some embodiments of the present application, there is provided a capnographic system, said system having one or more processors configured to receive an initial capnographic measurement from a breath monitoring device, at least when said breath monitoring device is attached to a patient; receive a primary value of at least one attribute, other than said initial capnographic measurement, characteristic of said patient; receive, from a database containing at least one set of secondary values corresponding to primary values of said at least one attribute, a secondary value assigned to the primary value of said at least one attribute; and calculate, based on a combination of said initial capnographic measurement and said secondary value, a refined capnographic measurement.

According to some embodiments, the system may also include the breath monitoring device having a sensor configured for obtaining capnographic measurements from the patient, and a display configured for displaying at least one of said initial capnographic measurement and said refined capnographic measurement. The capnographic measurements can be displayed in the form of a chart or a diagram that reflects the patient's CO₂ levels and exhaling pattern.

It is appreciated that there may be considerable variance between patients, dependent on age, weight, sex etc. In particular, any human patient has a certain volume of ‘dead space’ within his/her entire respiratory system, which may vary considerably between different groups of patients (e.g. men, women, children etc.). Thus, while a trained professional (e.g. a doctor) may be able to read a capnographic diagram and deduce from it details regarding the medical condition of the patient, consideration of attributes characteristic of the patient and/or the group to which the patient belongs can have a significant effect on the analysis of the diagram.

According to some embodiments, a database can first be constructed based on empirical or historical data. For example, the database can be constructed by performing a plurality of capnographic measurements on different patients from different groups, then grouping the characteristic attributes for each group. Each such group yields a set of baseline measurements, which may be average measurements unique to that group of patients. From these baseline measurements of each such group of attributes, at least one set of secondary values may be constructed. For example, the database can contain a first set of secondary values having a first secondary value associated with a first group of patients (e.g. women), a second secondary value associated with a second group (e.g. children), etc.

It should be noted that the grouping of patients does not have to be performed according to age or biological sex. In accordance with some embodiments, the grouping can be performed based on various factors or attributes, including the average volume of ‘dead space’ in patients.

According to some embodiments, in operation, when a patient is attached to the monitoring system, the processor receives information about the patient from two independent sources: capnographic measurements obtained by the sensor attached to the patient; and attributes about the patient (e.g. his biological sex, his age, his weight etc.) which are provided by other means (e.g. manual input).

According to some embodiments, once the capnographic measurements are received by the processor, they are combined, based on said attributes, with the corresponding secondary value(s) associated with that group of patients. For example, if a child is attached to the monitoring system, and an input is provided that the patient is a child, the processor can be configured to combine the capnographic measurements obtained from the child with a respective secondary value associated with children, thereby providing a more refined (e.g., accurate, patient-specific) capnographic data.

According to some embodiments, the at least one set of secondary values can be a list of coefficients, each pertaining to a different attribute characteristic of the patient. For example, as shown in Table 1, the columns labeled V_{A}, V_{W}, V_{M}, and V_{H} each represent a set of secondary values for a specific type of attribute (i.e., age, weight, medical condition, gender, height), and each of these columns contains multiple coefficients that may correspond to a specific attribute that is characteristic of the patient (e.g., the patient's age, weight, medical condition, gender, height):

TABLE 1

| Age | V_{A} | Weight | V_{W} | Medical condition | V_{M} | Sex | V_{S} | Height | V_{H} |
|---|---|---|---|---|---|---|---|---|---|
| 0-8 yr | α₁ | 0-20 kg | β₁ | Healthy | γ₁ | Male | δ₁ | 50-100 cm | ε₁ |
| 8-18 yr | α₂ | 20-40 kg | β₂ | Mildly ill | γ₂ | Female | δ₂ | 100-150 cm | ε₂ |
| 18-40 yr | α₃ | 40-60 kg | β₃ | Post surgery | γ₃ | | | 150-200 cm | ε₃ |
| 40-60 yr | α₄ | 60-80 kg | β₄ | | | | | 200-230 cm | ε₄ |
| 60-80 yr | α₅ | 80-100 kg | β₅ | | | | | | |

It is important to note that each column labeled V_{A}, V_{W}, V_{M}, and V_{H} in the above table represents a set of secondary values. Some sets may contain merely two or three secondary values (e.g. sex), while others can be broken down into a plurality of values (e.g. age, weight etc.).

According to some embodiments, any patient attached to the capnographic analysis system can contribute to the database. For example, each time a patient is attached to the system, the measurements obtained from the patient may be used to update the database, such as by storing the measurements and attributes characteristic of the patient in the database as a set. The more sets of historical and empirical data the database contains, the more accurate the baseline measurements it provides.

According to some embodiments, the one or more processors are configured to receive capnographic measurements from a breath monitoring device, at least when attached to a patient; receive at least one attribute characteristic of said patient; access the database and refine, based on the initial capnographic measurement and the at least one attribute, the set of secondary values stored therein. According to some embodiments, the one or more processors may include a dedicated processor (i.e., separate from the processor that calculates the refined capnographic measurement for the patient) that is configured to carry out these steps to refine the set of secondary values stored therein. In this way, the database can be a constantly growing and adapting entity, continuously collecting capnographic data about patients and refining its set of secondary values.

According to some embodiments, the capnographic system can include the processor, the sensor, and the display, while being remotely connected (e.g., wirelessly connected, such as via respective wireless transceivers) to the database. In some embodiments, two or more capnographic systems can be connected to a common database in order to receive data (e.g., secondary values) therefrom. In some embodiments, a plurality of capnographic systems can provide data (e.g., capnographic measurements from a patient and attributes characteristic of the patient) to the common database.

According to some embodiments, in operation, the capnographic system obtains the capnographic measurements from the patient, and obtains therefrom an attribute of interest, for example, the breathing volume, which can be designated as ΔT. One example of calculating ΔT follows the formula ΔT=DS/RT, where DS designates the dead space and RT designates the rise time. Thereafter, an operator (e.g., via manual inputs), or alternatively the system itself (e.g., by accessing patient records from a storage device or data from other sensors, such as a scale), inputs data about the patient (height, weight, sex etc.) to the processor, and the processor accesses the database of secondary values and chooses therefrom the corresponding secondary value(s), designated as K in the following equation and constituting a correction factor (e.g. a combination of coefficients or secondary values). From these two attributes, the effective volume V can be calculated, and the final equation can be, for example, V=X*K*ΔT, where λ is an optional correction factor that may be utilized in various equations disclosed herein.

For example, based on the above Table 1, if the patient is a healthy woman in her late 30s, with a height of 160 centimeters (cm) and a weight of 50 kilograms (kg), the equation to calculate the effective volume V will be V=λ*α₃*β₃*γ₁*δ₂*ε₃*ΔT.

The unique combination of coefficients relating to the attributes of that specific patient enable a more accurate and refined capnographic measurement. It is appreciated that the refined measurement is not restricted to an effective volume (V), and can also include a plurality of other capnographic attributes. For example, the processor may utilize the following equation V_{flow}=λ*K*DS/RT, where V_{flow} [liters/minutes or Lpm] is the calculated volume of exhaled breath or effective volume; K is a correction factor (e.g. one or more coefficients or secondary values); λ is another correction factor (e.g., to determine the linearity of the general equation ≅(2-[(RT [milliseconds])/(100 [milliseconds])]); DS [milliliters or ml] is the anatomic dead space, which is the volume of gas within the conducting zone, and includes the trachea, bronchus, bronchioles, and terminal bronchioles; it is approximately 2 ml/kg in the upright position. Therefore, the anatomic dead space is 156±28 ml in adults. Furthermore, the DS is composed of the volume of both the upper respiratory tract (including the nasal cavity, pharynx and larynx) and the lower respiratory tract (including the trachea, primary bronchi and lungs); and RT [milliseconds] defines the rise time by plotting CO₂ concentration against expired volume, including:
Phase I—CO₂ free portion of the tidal volume;
Phase II (Rise time)—transition between airway and alveolar; and
Phase III (EtCO₂)—CO₂ rich gas.

The above described embodiments enable obtaining the effective volume of CO₂ that is breathed by the patient using the capnographic measurement, while eliminating the need for an additional breathing monitor which calculates the actual volume of CO₂ breathed (requiring additional equipment and software).

It should be noted that the above mentioned attributes (weight, height etc.) all effect the DS parameter in the equation, each to its own degree. It is appreciated that some of the attributes can hold a greater weight in affecting the value of DS than others. In addition, in accordance with some embodiments, an initial DS can be determined according to one of the attributes (based on the database), and each of the other attributes can either increase or decrease the initial value DS.

For example, the initial value of DS can be assigned according to the age of the patient, i.e. for each primary value of age, the database can assign a corresponding value of DS. The values of the other attributes will affect the assigned value of DS by either increasing or decreasing it. Reverting to the previously discussed table, the a for each of the age groups designates an initial value of DS, wherein the equation may be the following V=X*β₃*γ₁*δ₂*ε₃*ΔT_{(α3)} in which ΔT_{(α3)}=DS_{(α3)}/RT, where the value of DS corresponds to the age group of patients who are 18 to 40 years old.

It should be noted that for each primary value of an attribute chosen for setting an initial value for DS, the secondary values of the remaining attributes should be properly associated with the above chosen primary value of the initial DS.

Using the above example, assuming age was chosen as the main attribute for defining the initial DS, and for a patient who is thirty years old, the parameter α3 was chosen for setting an initial DS such that DS_{(α3)}=M (ml). The remaining attributes will either increase this value or decrease it, compared to the standard value of the corresponding attribute for this specific age. Thus, a weight of 0-20 kg will lower the value of DS_{(α3)} (i.e. β₁<1), a weight of 40-60 kg will not affect the value of DS_{(α3)} too much (i.e. β₃≈1), and a weight of 80-100 kg will increase the value of DS_{(α3)} (i.e. β₅>1).

However, if the same attribute (age) is chosen for determining the initial DS in a patient who is four years old, any weight above 20 kg will cause an increase in the initial value of DS (i.e. β₂, β₃, β₄, β₅>1).

Thus, the database, in accordance with some embodiments, can hold a plurality of sets of secondary values, each set corresponding to a value of an attribute which is chosen to define the initial DS of the patient. In other words, the set of secondary values β₁ through β₅ for determining the DS based on an age group corresponding to α₁, may be different that the set of secondary values β₁ through β₅ for determining the DS based on an age group corresponding to α₂ and so on for each attribute and value.

It should be noted that the database does not necessarily have to contain a separate set of secondary values for each and every one of the values of an attribute according to which the initial DS is assigned. More specifically, some attributes can have a similar effect on the initial value of DS, regardless of the attribute according to which it was assigned. As an example, the secondary values of the ‘medical condition’ attribute can have a constant effect on the initial DS values, in the sense that sickness may reduce the value of DS.

It is also appreciated that, once the effective volume is obtained, it can be compared (e.g., by the processor) to standard charts and databases to determine if there is something wrong with the patient. In addition, the interesting and clinically useful portions of the signal may be the rise time and the fall time, and possibly the integral under the entire curve of the signal. Accordingly, in some embodiments, the processor may be configured to determine the rise time, the fall time, and/or the integral to assess and/or to provide an output (e.g., audible or visual alarm or message) indicative of the status of the patient.

According to some embodiments, the system may include an auxiliary database containing data regarding a tubing that may be used in conjunction with the breath monitoring device and representing the volume of the system. Thus, once a specific model of the device is used, an operator can either manually introduce the make and model to the processor, or the processor can recognize the make and model of the device automatically (e.g., upon connection), and indicate to the processor which value to select and use from the auxiliary database (i.e., the auxiliary database may store multiple values that each correspond to various tubing and/or breath monitoring devices that may be used with the system). Under such a configuration, the overall volume of the tubing, equipment, and patient are taken into account in calculating the refined capnographic measurement. Consideration of the volume of the tubing can assist in a more refined calculation of the effective breathable volume, since the volume of the tubing can now be properly deducted from the capnographic data. For example, such deduction can be performed with respect to the rise and fall time of the capnographic measurement by the processor.

According to some embodiments, there is provided one or more processors constituting a part of a capnographic system, said one or more processors being configured to: receive an initial capnographic measurement from a breath monitoring device, at least when said breath monitoring device is attached to a patient; receive a primary value of at least one attribute, other than said capnographic measurement, characteristic of said patient; receive, from a database containing at least one set of secondary values assigned to primary values of said at least one attribute, a secondary value corresponding to the primary value of said at least one attribute; and calculate, based on a combination of said initial capnographic measurement and said secondary value, a refined capnographic measurement.

### BRIEF DESCRIPTION OF THE DRAWINGS

Various embodiments are illustrated by way of example in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIG. 1A (prior art) is a schematic illustration of the human respiratory tract;

FIG. 1B (prior art) is a schematic capnographic diagram showing CO₂ measurements over time during exhaling;

FIG. 2 illustrates an embodiment of a capnography system that is configured to monitor a CO₂ level in a patient;

FIG. 3A is a graph of a CO₂ level of a patient having a first set of characteristics;

FIG. 3B is a graph of a CO₂ level of another patient having a second set of characteristics;

FIG. 3C is a graph of a CO₂ level of another patient having a third set of characteristics; and

FIG. 3D is a graph of a CO₂ level of another patient having a fourth set of characteristics.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS

One or more specific embodiments of the present disclosure will be described below. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but may nevertheless be a routine undertaking of design, fabrication, and manufacture for those of ordinary skill having the benefit of this disclosure. Further, the current embodiments may be implemented by one or more computer-processors that implement one or more machine-readable instructions stored on a tangible, non-transitory, machine-readable medium and/or by specialized circuitry designed to implement the discussed features.

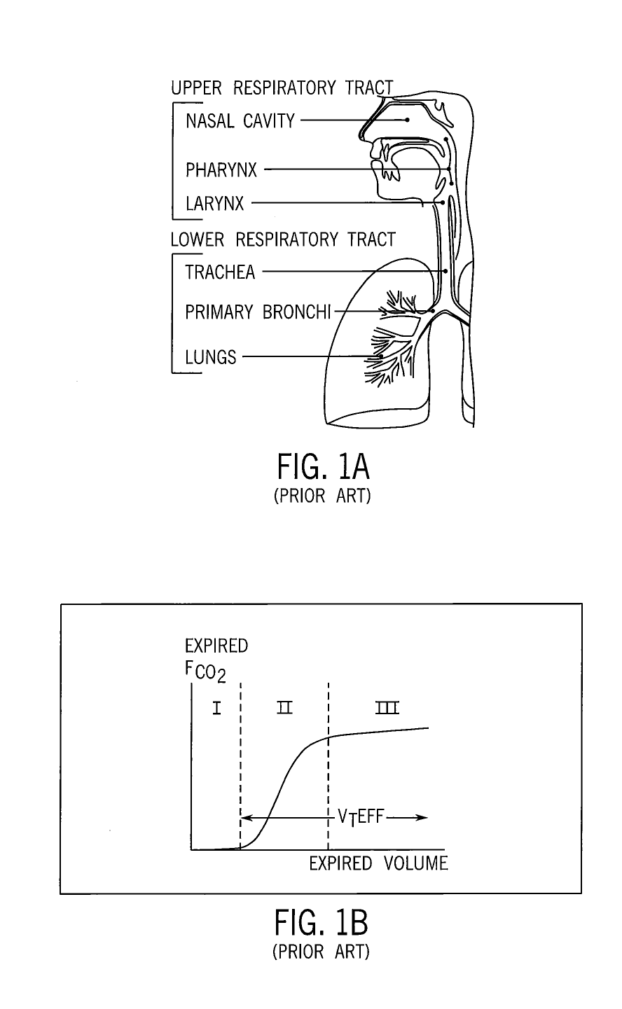

Attention is first drawn to FIG. 1A in which the human respiratory system is shown. The respiratory system comprises the upper respiratory tract, including the nasal cavity, pharynx, and larynx, and the lower respiratory tract including the trachea, primary bronchi, and lungs. This volume is also referred herein as ‘dead space’. It is appreciated that the volume of the respiratory system varies from one person to another, and is affected by various characteristics, such as weight, height, age, gender etc. Turning now to FIG. 1B, an example of a standard capnographic diagram is shown, illustrating the CO₂ levels in a patient during inhaling, represented by three phases: Phase I—CO₂ free portion of the tidal volume; Phase II (Rise time)—transition between airway and alveolar; and Phase III (end-tidal CO₂ [EtCO₂])—CO₂ rich gas.

In some methods, the volume of breathed air may be calculated based on the area below the measured curve during the phase II. However, due to the variance in dead space between patients, applying this method may yield inaccurate results.

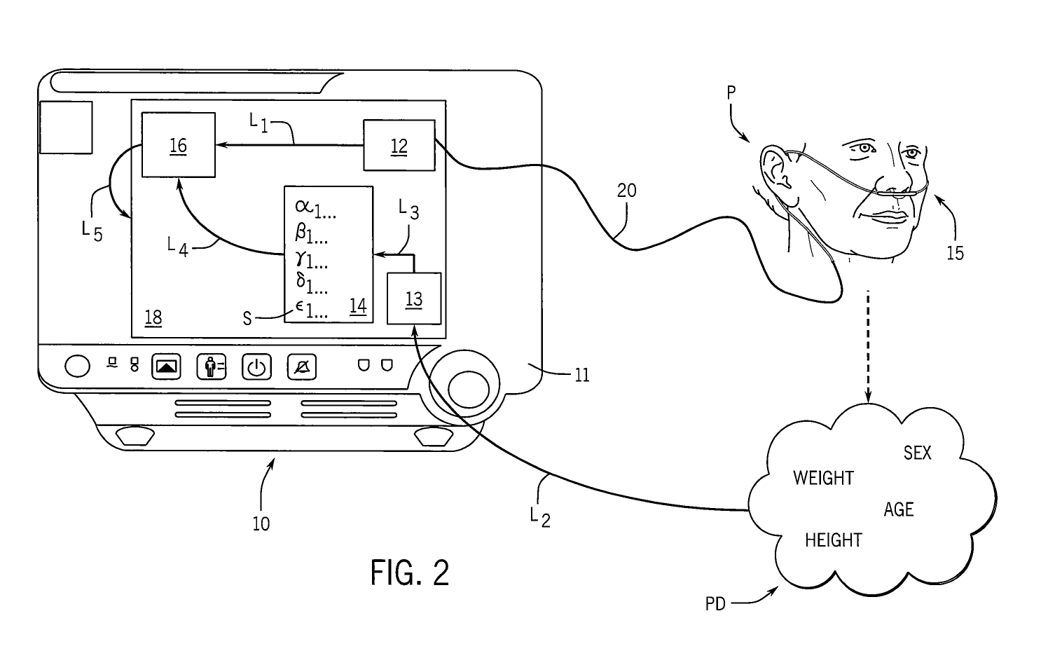

Turning now to FIG. 2, an embodiment of a capnography system 10 is shown. The system 10 is configured for monitoring, inter alia, CO₂ level in a patient P. In the illustrated embodiment, the system 10 includes a measurement module 12, an input module 13, a database 14, a processor 16, and a display 18, some or all of which may be supported or housed within a monitor 11 (e.g., a patient monitor).

The patient P has a cannula or mask 15 (e.g., sensor) fitted to his/her face, which is, in turn, connected to the system 10 via appropriate tubing 20. This connection allows the system 10 to measure and monitor the CO₂ levels of the patient. In particular, these measurements are provided via connection L₁ (e.g., electronic and/or wireless connection) to the processor 16, which derives the rise time RT from the measurements.

In addition, one or more various attributes of the patient P, such as height, age, weight etc., are collected (e.g. accessed from patient records and/or by an operator of the system 10) and are input into the input module 13 via connection L₂ (e.g., via an interface on the system 10 itself). Thus, the input may include primary values of the different attributes of the patient P. The input module 13 is communicatively coupled, via connection L₃ (e.g., electronic and/or wireless connection), with a database 14 that stores one or more sets of secondary values S, each set corresponding to a different type of attribute. For example, a set S_{HEIGHT} for height values from the input module 13, a set S_{WEIGHT} for weight values from the input module 13 etc.

The database 14 can store data in the form of a table, such as Table 1, which is reproduced below:

TABLE 1

| Age | V_{A} | Weight | V_{W} | Medical condition | V_{M} | Sex | V_{S} | Height | V_{H} |
|---|---|---|---|---|---|---|---|---|---|
| 0-8 yr | α₁ | 0-20 kg | β₁ | Healthy | γ₁ | Male | δ₁ | 50-100 cm | ε₁ |
| 8-18 yr | α₂ | 20-40 kg | β₂ | Mildly ill | γ₂ | Female | δ₂ | 100-150 cm | ε₂ |
| 18-40 yr | α₃ | 40-60 kg | β₃ | Post surgery | γ₃ | | | 150-200 cm | ε₃ |
| 40-60 yr | α₄ | 60-80 kg | β₄ | | | | | 200-230 cm | ε₄ |
| 60-80 yr | α₅ | 80-100 kg | β₅ | | | | | | |

The processor 16 is configured to select and/or to receive, from the database 14, via a connection L₄ (e.g., electronic and/or wireless connection), a secondary value corresponding to the primary value provided by the input module 13. For example, if the input module 13 receives an input Height=110 cm (e.g., the primary value), the processor 16 will select and/or receive from the database 14 the value ε2 (e.g., the secondary value). It is noted that the processor 16 may receive a secondary value for each of the primary values of the attributes introduced, so the processor 16 may receive one or more secondary values simultaneously and/or at approximately the same time for use during the monitoring session for the patient. Thus, for a patient who is a healthy woman in her late 30s, with a height of 160 cm, and a weight of 50 kg, the secondary values provided to the processor 16 using Table 1 will be: α₃, β₃, γ₁, δ₂, and ε₃.

The processor 16 is configured to combine the measurement of RT obtained directly from the patient P with the DS value calculated based on the secondary values in order to calculate the effective volume V_{E}, and may output the result to the display 18 via connection L₅. The combination of secondary values can be used to calculate the effective volume by the following formula, for example:

$${V}_{E} = λ * {α}_{3} * {β}_{3} * {γ}_{1} * {δ}_{2} * {ɛ}_{3} * \frac{DS}{RT}$$

In some embodiments disclosed herein, the processor 16 assigns an initial value of DS based on the age of the patient, and the remainder of the attributes affect this initial value by either increasing or decreasing this initial value. Thus, the formula can be the following:

$${V}_{E} = λ * {β}_{3} * {γ}_{1} * {δ}_{2} * {ɛ}_{3} * \frac{{DS}_{( α 3 )}}{RT}$$

Under the above example, the parameter α₃ determines an initial value for DS itself, rather than being used as a coefficient. However, it is appreciated that in accordance with other examples, other attributes can be used for setting an initial value for DS, wherein the equations vary correspondingly, e.g.:

$$DS based on height \text{:} {V}_{E} = λ * {α}_{3} * {β}_{3} * {γ}_{1} * {δ}_{2} * \frac{{DS}_{( ɛ 3 )}}{RT}$$

$$DS based on Weight \text{:} {V}_{E} = λ * {α}_{3} * {γ}_{1} * {δ}_{2} * {ɛ}_{3} * \frac{{DS}_{( β 3 )}}{RT}$$

$$DS based on S ex \text{:} {V}_{E} = λ * {α}_{3} * {β}_{3} * {γ}_{1} * {ɛ}_{3} * \frac{{DS}_{( δ 2 )}}{RT}$$

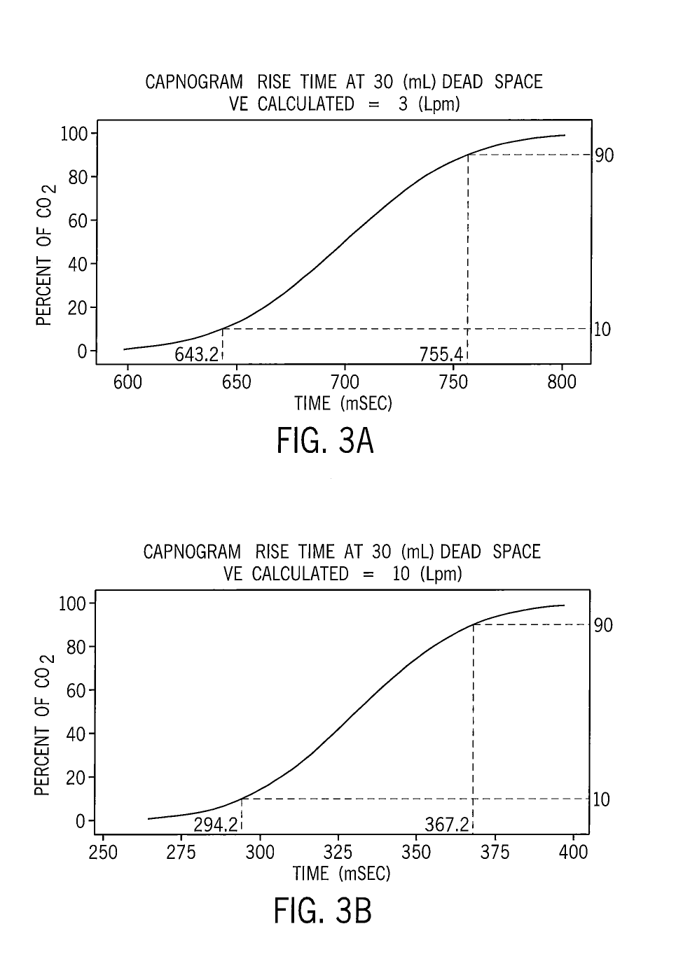

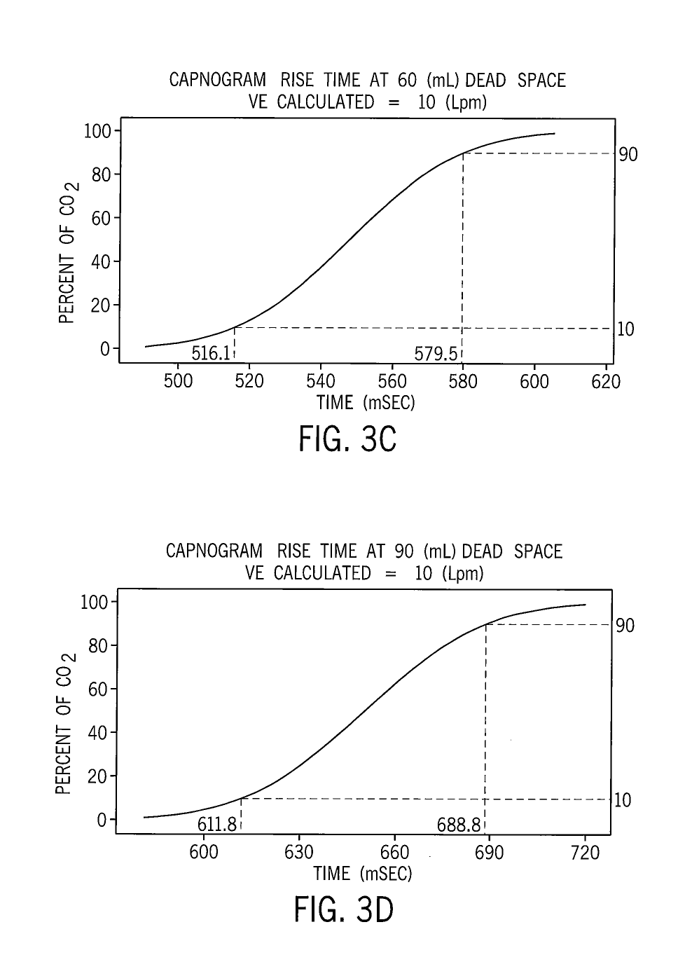

Turning now to FIGS. 3A to 3D, different examples of capnographic diagrams for different patients are shown along with a corresponding calculation of the effective volume V_{E}. It should be appreciated that the values (e.g., ranges and/or coefficient values) shown in the following tables are provided to facilitate discussion and the values may vary in practice. Furthermore, it should be appreciated that other types of attributes (e.g., body mass index [BMI], torso measurements, or the like) may be included in the table and may be utilized to determine the effective volume.

Referring now to FIG. 3A, a diagram is shown for a healthy woman in her 30's, weighing 80 kg with a height of 140 cm. In order to determine the effective volume, an initial value of DS is assigned according to the woman's age, based on the data stored in the database, such as:

TABLE 3

| Age (years) | DS (ml) |
|---|---|
| 0-8 | 100 |
| 8-18 | 120 |
| 18-40 | 155 |
| 40-60 | 145 |
| 60-80 | 135 |

Once the initial DS has been determined (in this case—DS_{(α3)}), the remainder of the secondary values of the other attributes are used to adjust (e.g., increase/decrease) the initial value of DS_{(α3)}, and resulting value of the effective volume V_{E}.

TABLE 4

| | | Medical | | | | | |
|---|---|---|---|---|---|---|---|
| Weight | V_{W} | condition | V_{M} | Sex | V_{S} | Height | V_{H} |
| 0-20 kg | 0.5 | Healthy | 1.2 | Male | 1.2 | 50-100 cm | 0.85 |
| 20-40 kg | 0.85 | Mildly ill | 0.8 | Female | 1 | 100-150 cm | 1 |
| 40-60 kg | 1 | Post | 0.7 | | | 150-200 cm | 1.05 |
| | | surgery | | | | | |
| 60-80 kg | 1.15 | | | | | 200-230 cm | 1.3 |
| 80-100 kg | 2 | | | | | | |

The parameter λ (correction factor) is calculated, as previously mentioned, as:

λ≅(2−[(RT[mSec])/(100[mSec])])

The rise time in the present example is RT=755.4−643.2=112.2. Thus, the equation for λ is:

λ≅[2−(112.2/100)]=0.878

Using Table 4, the effective volume V_{E} is calculated based on the following formula:

$${V}_{E} = λ \times {β}_{5} \times {γ}_{1} \times {δ}_{2} \times {ɛ}_{3} \times \frac{{DS}_{α 3}}{RT} = 0.878 \times 2 \times 1.2 \times 1 \times 1 \times \frac{155}{112.2} = 2.91 [ Lpm ]$$

Referring now to FIG. 3B, a diagram is shown for a healthy man in his 30's, weighing 120 kg with a height of 200 cm. In order to determine the effective volume, an initial value of DS is assigned according to the man's age, based on the data stored in the database, such as in Table 3, which is reproduced below:

TABLE 3

| Age (years) | DS (ml) |
|---|---|
| 0-8 | 100 |
| 8-18 | 120 |
| 18-40 | 155 |
| 40-60 | 145 |
| 60-80 | 135 |

Once the initial DS has been determined (in this case—DS_{(α4)}), the remainder of the secondary values of the other attributes are used to adjust (e.g., increase/decrease) the initial value of DS_{(α4)}, and resulting value of the effective volume V_{E}.

TABLE 4

| | | Medical | | | | | |
|---|---|---|---|---|---|---|---|
| Weight | V_{W} | condition | V_{M} | Sex | V_{S} | Height | V_{H} |
| 0-20 kg | 0.5 | Healthy | 1.2 | Male | 1.1 | 50-100 cm | 0.85 |
| 20-40 kg | 0.85 | Mildly ill | 0.8 | Female | 1 | 100-150 cm | 1 |
| 40-60 kg | 1 | Post | 0.7 | | | 150-200 cm | 1.2 |
| | | surgery | | | | | |
| 60-80 kg | 1.15 | | | | | 200-230 cm | 1.5 |
| 80-100 kg | 2 | | | | | | |

The parameter λ (correction factor) is calculated, as previously mentioned, as:

λ≅(2−[(RT[mSec])/(100[mSec])])

The rise time in the present example is RT=367.2−294.2=73. Thus, the equation for λ is:

λ≅[2−(73/100)]=1.27

Using Table 4, which is reproduced above, the effective volume V_{E} is calculated based on the following formula:

$${V}_{E} = λ \times {β}_{5} \times {γ}_{1} \times {δ}_{2} \times {ɛ}_{3} \times \frac{{DS}_{α 3}}{RT} = 1.27 \times 2 \times 1.2 \times 1.1 \times 1.5 \times \frac{155}{73} = 10.6 [ Lpm ]$$

Referring now to FIG. 3C, a diagram is shown for a healthy 17 year old male teenager, weighing 85 kg with a height of 190 cm. In order to determine the effective volume, an initial value of DS is assigned according to the man's age, based on the data stored in the database, such as in Table 3, which is reproduced below:

TABLE 3

| Age (years) | DS (ml) |
|---|---|
| 0-8 | 100 |
| 8-18 | 120 |
| 18-40 | 155 |
| 40-60 | 145 |
| 60-80 | 135 |

Once the initial DS has been determined (in this case—DS_{(α2)}), the remainder of the secondary values of the other attributes are used to adjust (e.g., increase/decrease) the initial value of DS_{(α2)}, and resulting value of the effective volume V_{E}.

TABLE 4

| | | Medical | | | | | |
|---|---|---|---|---|---|---|---|
| Weight | V_{W} | condition | V_{M} | Sex | V_{S} | Height | V_{H} |
| 0-20 kg | 0.5 | Healthy | 1.2 | Male | 1.1 | 50-100 cm | 0.85 |
| 20-40 kg | 0.85 | Mildly ill | 0.8 | Female | 1 | 100-150 cm | 1 |
| 40-60 kg | 1 | Post | 0.7 | | | 150-200 cm | 1.2 |
| | | surgery | | | | | |
| 60-80 kg | 1.15 | | | | | 200-230 cm | 1.5 |
| 80-100 kg | 2 | | | | | | |

The parameter λ (correction factor) is calculated, as previously mentioned, as:

λ≅(2−[(RT[mSec])/(100[mSec])])

The rise time in the present example is RT=579.5−516.1=63.4. Thus, the equation for λ is:

λ≅[2−(63.4/100)]=1.366

Using Table 4, which is reproduced above, the effective volume V_{E} is calculated based on the following formula:

$${V}_{E} = λ \times {β}_{5} \times {γ}_{1} \times {δ}_{2} \times {ɛ}_{3} \times \frac{{DS}_{α 3}}{RT} = 1.366 \times 2 \times 1.2 \times 1.1 \times 1.5 \times \frac{120}{63.4} = 10.23 [ Lpm ]$$

Referring now to FIG. 3D, a diagram is shown for a mildly ill man in his 40's, who is extremely obese (weighing 200 kg) with a height of 160 cm. In order to determine the effective volume, an initial value of DS is assigned according to the man's age, based on the data stored in the database, such as in Table 3, which is reproduced below:

TABLE 3

| Age (years) | DS (ml) |
|---|---|
| 0-8 | 100 |
| 8-18 | 120 |
| 18-40 | 155 |
| 40-60 | 145 |
| 60-80 | 135 |

Once the initial DS has been determined (in this case—DS_{(α4)}), the remainder of the secondary values of the other attributes are used to adjust (e.g., increase/decrease) the initial value of DS_{(α4)}, and resulting value of the effective volume V_{E}.

TABLE 4

| | | Medical | | | | | |
|---|---|---|---|---|---|---|---|
| Weight | V_{W} | condition | V_{M} | Sex | V_{S} | Height | V_{H} |
| 0-20 kg | 0.5 | Healthy | 1.2 | Male | 1.2 | 50-100 cm | 0.85 |
| 20-40 kg | 0.85 | Mildly ill | 0.8 | Female | 1 | 100-150 cm | 1 |
| 40-60 kg | 1 | Post | 0.7 | | | 150-200 cm | 1.05 |
| | | surgery | | | | | |
| 60-80 kg | 1.15 | | | | | 200-230 cm | 1.3 |
| 80-100 kg | 2 | | | | | | |
| Obese | 3 | | | | | | |

The parameter λ (correction factor) is calculated, as previously mentioned, as:

λ≅(2−[(RT[mSec])/(100[mSec])])

The rise time in the present example is RT=688.8−611.8=77. Thus, the equation for λ is:

λ≅[2−(77/100)]=1.23

Using Table 4, which is reproduced above, the effective volume V_{F} is calculated based on the following formula:

$${V}_{E} = λ \times {β}_{5} \times {γ}_{1} \times {δ}_{2} \times {ɛ}_{3} \times \frac{{DS}_{α 3}}{RT} = 1.23 \times 3.5 \times 0.8 \times 1.2 \times 1.3 \times \frac{145}{77} = 10.117 [ Lpm ]$$

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope. Those skilled in the art to which this disclosure pertains will readily appreciate that numerous changes, variations, and modifications can be made without departing from the scope of the disclosure, mutatis mutandis.

1. A capnographic system comprising one or more processors configured to:
receive an initial capnographic measurement from a breath monitoring device, at least when said breath monitoring device is attached to a patient;
receive a primary value of at least one attribute, other than said initial capnographic measurement, characteristic of said patient;
receive, from a database containing at least one set of secondary values assigned to primary values of said at least one attribute, a secondary value corresponding to the primary value of said at least one attribute; and
calculate, based on a combination of said initial capnographic measurement and said secondary value, a refined capnographic measurement.

2. The system of claim 1, wherein the system comprises the breath monitoring device comprising a sensor configured to obtain the initial capnographic measurement from the patient.

3. The system of claim 1, wherein the system comprises a display configured to display at least one of said initial capnographic measurement and said refined capnographic measurement.

4. The system of claim 3, wherein the at least one of said initial capnographic measurement and said refined capnographic measurement is displayed in the form of a chart or a diagram reflecting the patient's CO₂ levels and exhaling pattern.

5. The system of claim 1, wherein said at least one attribute is chosen from a group comprising at least biological sex, age, weight, height, and medical condition.

6. The system of claim 1, wherein the at least one set of secondary values comprises a list of coefficients, each pertaining to a different attribute characteristic of the patient.

7. The system of claim 1, wherein the initial capnographic measurement obtained from the patient is used to expand said database.

8. The system of claim 1, wherein the one or more processors are configured to:
receive capnographic measurements from the breath monitoring device, at least when attached to the patient;
receive at least one attribute characteristic of said patient; and
access the database and refine, based on the capnographic measurements and the at least one attribute, the set of secondary values stored therein.

9. The system of claim 1, wherein the capnographic system comprises the one or more processors, the sensor, and the display, and the capnographic system is remotely connected to the database.

10. The system of claim 9, wherein at least the capnographic system and another capnographic system are connected to the database in order to receive data therefrom and/or to provide data thereto.

11. The system of claim 1, wherein said one or more processors are configured to:
receive another primary value for another attribute characteristic of said patient;
receive, from the database, another secondary value corresponding to the another primary value; and
calculate the refined capnographic measurement based on the combination of the initial capnographic measurement, the secondary value, and the another secondary value.

12. The system of claim 1, wherein said secondary value is used as a coefficient in calculating an effective volume using the formula V=λ*K*ΔT, where V is the effective volume, K is the coefficient, λ is a correction factor, and ΔT is a breathing volume

13. The system of claim 12, wherein said coefficient reflects a variance in patient dead space.

14. The system of claim 13, wherein said patient dead space comprises both an upper respiratory tract comprising a nasal cavity, a pharynx, and a larynx, and a lower respiratory tract comprising a trachea, a primary bronchi, and lungs of the patient.

15. The system of claim 1, wherein the system comprises an auxiliary database comprising data regarding a tubing used in conjunction with the breath monitoring device, and representing a volume of the system.

16. The system of claim 15, wherein the one or more processors are configured to access the data from the auxiliary database and to calculate the refined capnographic measurement based on said initial capnographic measurement, said secondary value, and the data representing the volume of the system.

17. One or more processors configured to be used as part of a capnographic system, said one or more processors being configured to:
receive an initial capnographic measurement from a breath monitoring device, at least when said breath monitoring device is attached to a patient;
receive a primary value of at least one attribute, other than said initial capnographic measurement, characteristic of said patient;
receive, from a database containing at least one set of secondary values assigned to primary values of said at least one attribute, a secondary value corresponding to the primary value of said at least one attribute; and
calculate, based on a combination of said initial capnographic measurement and said secondary value, a refined capnographic measurement.
